SEQUENCE LISTING (34 sequences)

SEQ ID NO: 1 - DNA, 41 bp, Homo sapiens
taatacgactcactatagggcctcttgctcctgctggcttt

SEQ ID NO: 2 - DNA, 42 bp, Homo sapiens
taatacgactcactatagggatgggttgggggtgcagctgtt

SEQ ID NO: 3 - DNA, 506 bp, Homo sapiens
cctcttgctcctgctggctttcggccacagcacctatggggctgaatgcttcccggcctgcaacccccaaaatggattctgcgaggatgacaatgtttgcaggtgccagcctggctggcagggtcccctttgtgaccagtgcgtgacctctcccggctgccttcacggactctgtggagaacccgggcagtgcatttgcaccgacggctgggacggggagctctgtgatagagatgttcgggcctgctcctcggccccctgtgccaacaacaggacctgcgtgagcctggacgatggcctctatgaatgctcctgtgcccccgggtactcgggaaaggactgccagaaaaaggacgggccctgtgtgatcaacggctccccctgccagcacggaggcacctgcgtggatgatgagggccgggcctcccatgcctcctgcctgtgcccccctggcttctcaggcaatttctgcgagatcgtggccaacagctgcacccccaacccat

SEQ ID NO: 4 - DNA, 40 bp, Homo sapiens
taatacgactcactatagggagcagcttgggctcgagaag

SEQ ID NO: 5 - DNA, 40 bp, Homo sapiens
taatacgactcactatagggccctttgtgttcccaattcc

SEQ ID NO: 6 - DNA, 423 bp, Homo sapiens
agcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtgccccaggctatgggagccctcacttcactgcactgtactcctcggtccctttccctgagggggaagcctttccccctgtctctgtcaccactctgggctctcccttgcattcaaactgaggtgcctgcctgcccttctaggaatgggggacagggggaggggaggagctagggaaagaaaacctggagtttgtgccagggtttttggattaagttcttcattcactaaggaaggaattgggaacacaaaggg

SEQ ID NO: 7 - DNA, 43 bp, Homo sapiens
aattataatacgactcactatacgtgggctgcggggtgctgct

SEQ ID NO: 8 - DNA, 42 bp, Homo sapiens
aattataatacgactcactatatgcaggaggcgatcatgagc

SEQ ID NO: 9 - DNA, 425 bp, Homo sapiens
cgtgggctgcggggtgctgctgtcccgcaagcgccggcggcagcatggccagctctggttccctgagggcttcaaagtgtctgaggccagcaagaagaagcggcgggagcccctcggcgaggactccgtgggcctcaagcccctgaagaacgcttcagacggtgccctcatggacgacaaccagaatgagtggggggacgaggacctggagaccaagaagttccggttcgaggagcccgtggttctgcctgacctggacgaccagacagaccaccggcagtggactcagcagcacctggatgccgctgacctgcgcatgtctgccatggcccccacaccgccccagggtgaggttgacgccgactgcatggacgtcaatgtccgcgggcctgatggcttcaccccgctcatgatcgcctcctgca

SEQ ID NO: 10 - DNA, 41 bp, Homo sapiens
taatacgactcactatagggtcgtgcaagagccagttaccc

SEQ ID NO: 11 - DNA, 41 bp, Homo sapiens
taatacgactcactatagggaatgtcatggccgcttcagag

SEQ ID NO: 12 - DNA, 537 bp, Homo sapiens
tcgtgcaagagccagttacccacccacaggtccccctacttcctgccaagcattccattgactgcctgtatggaacacatttgtcccagatctgagcattctaggcctgtttcactcactcacccagcatatgaaactagtcttaactgttgagcctttcctttcatatccacagaagacactgtctcaaatgttgtacccttgccatttaggactgaactttccttagcccaagggacccagtgacagttgtcttccgtttgtcagatgatcagtctctactgattatcttgctgcttaaaggcctgctcaccaatctttctttcacaccgtgtggtccgtgttactggtatacccagtatgttctcactgaagacatggactttatatgttcaagtgcaggaattggaaagttggacttgttttctatgatccaaaacagccctataagaaggttggaaaaggaggaactatatagcagcctttgctattttctgctaccatttcttttcctctgaagcggccatgacattccct

SEQ ID NO: 13 - DNA, 40 bp, Homo sapiens
taatacgactcactatagggtcctgtgcctgtggtagaga

SEQ ID NO: 14 - DNA, 42 bp, Homo sapiens
taatacgactcactatagggactgtggctgtagatgatgtga

SEQ ID NO: 15 - DNA, 438 bp, Homo sapiens
tcctgtgcctgtggtagagagccttcagttgaatggcggtggggacgtagcaatgcttgaacttacaggacagaatttcactccaaatttacgagtgtggtttggggatgtagaagctgaaactatgtacaggtgtggagagagtatgctctgtgtcgtcccagacatttctgcattccgagaaggttggagatgggtccggcaaccagtccaggttccagtaactttggtccgaaatgatggaatcatttattccaccagccttacctttacctacacaccagaaccagggccacggccacattgcagtgtagcaggagcaatccttccagccaattcaagccaggtgccccctaacgaatcaaacacaaacagcgagggaagttacacaaacgccagcacaaattcaaccagtgtcacatcatctacagccacagt

SEQ ID NO: 16 - DNA, 42 bp, Homo sapiens
taatacgactcactatagggagtagtgagagtgagagtaaca

SEQ ID NO: 17 - DNA, 43 bp, Homo sapiens
taatacgactcactatagggctctatacaagtctgtgccatgg

SEQ ID NO: 18 - DNA, 463 bp, Homo sapiens
agtagtgagagtgagagtaacaataaagaaaaaaaaatacaaaggaagaaaagaaagaaaaacaagtgttcagggcataacaacagtgattctgaagagaaggacaagtctaagaagagaaagcttcatgaagaactttctagcagtcaccataaccgggaaaaagccaaggaaaagcccaggttcttaaaacacgagagttctagggaggacagcaaatggagccattctgattctgacaaaaagtccagaacccataaacatagcccagagaagagaggctctgaaagaaaggaggggagcagcagaagccacggcagggaggaaaggagccggagaagccggagcagaagtcctggtagttacaagcaaagggagacaaggaaacgggcacagcgaaatcctggtgaagagcaaagcagaagaaatgacagcagaagccatggcacagacttgtatagag

SEQ ID NO: 19 - DNA, 24 bp, Homo sapiens
gcggccgcctttgtggttctgttc

SEQ ID NO: 20 - DNA, 21 bp, Homo sapiens
gccggcgcgtcctcctcttcc

SEQ ID NO: 21 - DNA, 20 bp, Homo sapiens
gccagaatgatgctacctgt

SEQ ID NO: 22 - DNA, 20 bp, Homo sapiens
tagagcagcaccaatggaac

SEQ ID NO: 23 - DNA, 25 bp, Homo sapiens
aagttacccccaagaggcaagtgtt

SEQ ID NO: 24 - DNA, 25 bp, Homo sapiens
aaggaaatgagaggccagaaggaga

SEQ ID NO: 25 - DNA, 20 bp, Homo sapiens
ggctgcccctcccactctcg

SEQ ID NO: 26 - DNA, 20 bp, Homo sapiens
cagcccgggccccaggatag

SEQ ID NO: 27 - DNA, 20 bp, Homo sapiens
tgagtggttcctgtactctg

SEQ ID NO: 28 - DNA, 20 bp, Homo sapiens
actcacactgggtaacacgg

SEQ ID NO: 29 - DNA, 24 bp, Homo sapiens
tacccagagcggcctatcattttt

SEQ ID NO: 30 - DNA, 24 bp, Homo sapiens
acgaagccggccaggaggaaggac

SEQ ID NO: 31 - DNA, 24 bp, Homo sapiens
tggcctgaggagcttgaatgtgac

SEQ ID NO: 32 - DNA, 24 bp, Homo sapiens
atcgcccagcaaaaatccaatgaa

SEQ ID NO: 33 - DNA, 23 bp, Homo sapiens
agaggagtggctgcaatgaggtc

SEQ ID NO: 34 - DNA, 18 bp, Homo sapiens
gcgcccggctgttttctt

(19) United States
(12) Patent Application Publication
(10) Pub. No.: US 2004/0053869 A1
(43) Pub. Date: Mar. 18, 2004

(54) Stem cell differentiation

(76) Inventors: Peter Andrews, Sheffield (GB); James Walsh, Sheffield (GB); Paul Gokhale, Sheffield (GB)

Correspondence Address: CROWELL & MORING LLP, INTELLECTUAL PROPERTY GROUP, P.O. BOX 14300, WASHINGTON, DC, 20044-4300, US

(21) Appl. No.: 10/344,928

(22) PCT Filed: Aug. 17, 2001
(86) PCT No.: PCT/GB01/03680

(30) Foreign Application Priority Data
Aug. 19, 2000 (GB) 0020396.8
Mar. 15, 2001 (GB) 0106329.6

(51) Int. Cl. A61K 48/00; C12N 5/08; C12N 15/63

(52) U.S. Cl. 514/44; 435/366; 435/455

(57) ABSTRACT

The invention relates to a method to modulate stem cell differentiation comprising introducing inhibitory RNA (RNAi) into a stem cell to ablate mRNA's which encode polypeptides which are involved in stem cell differentiation; RNAi molecules, DNA molecules encoding said RNAi molecules; and cells obtained by said method.

The invention relates to a method to modulate stem cell differentiation comprising introducing inhibitory RNA (RNAi) into a stem cell to ablate mRNA's which encode polypeptides which are involved in stem cell differentiation. Typically these mRNA's encode negative regulators of differentiation the removal of which promotes differentiation into a particular cell type(s).

A number of techniques have been developed in recent years which purport to specifically ablate genes and/or gene products. For example, the use of anti-sense nucleic acid molecules to bind to and thereby block or inactivate target mRNA molecules is an effective means to inhibit the production of gene products. This is typically very effective in plants where anti-sense technology produces a number of striking phenotypic characteristics. However, antisense is variable leading to the need to screen many, sometimes hundreds of, transgenic organisms carrying one or more copies of an antisense transgene to ensure that the phenotype is indeed truly linked to the antisense transgene expression. Antisense techniques, not necessarily involving the production of stable transfectants, have been applied to cells in culture, with variable results.

In addition, the ability to be able to disrupt genes via homologous recombination has provided biologists with a crucial tool in defining developmental pathways in higher organisms. The use of mouse gene “knock out” strains has allowed the dissection of gene function and the probable function of human homologues to the deleted mouse genes, (Jordan and Zant, 1998).

A much more recent technique to specifically ablate gene function is through the introduction of double stranded RNA, also referred to as inhibitory RNA (RNAi), into a cell which results in the destruction of mRNA complementary to the sequence included in the RNAi molecule. The RNAi molecule comprises two complementary strands of RNA (a sense strand and an antisense strand) annealed to each other to form a double stranded RNA molecule. The RNAi molecule is typically derived from exonic or coding sequence of the gene which is to be ablated.

Recent studies suggest that RNAi molecules ranging from 100-1000 bp derived from coding sequence are effective inhibitors of gene expression. Suprisingly, only a few molecules of RNAi are required to block gene expression which implies the mechanism is catalytic. The site of action appears to be nuclear as little if any RNAi is detectable in the cytoplasm of cells indicating that RNAi exerts its effect during mRNA synthesis or processing.

The exact mechanism of RNAi action is unknown although there are theories to explain this phenomenon. For example, all organisms have evolved protective mechanisms to limit the effects of exogenous gene expression. For example, a virus often causes deleterious effects on the organism it infects. Viral gene expression and/or replication therefore needs to be repressed. In addition, the rapid development of genetic transformation and the provision of transgenic plants and animals has led to the realisation that transgenes are also recognised as foreign nucleic acid and subjected to phenomena variously called quelling (Singer and Selker, 1995), gene silencing (Matzke and Matzke, 1998), and co-suppression (Stam et. al., 2000).

Initial studies using RNAi used the nematode Caenorhabditis elegans. RNAi injected into the worm resulted in the disappearance of polypeptides corresponding to the gene sequences comprising the RNAi molecule(Montgomery et. al., 1998; Fire et. al., 1998). More recently the phenomenon of RNAi inhibition has been shown in a number of eukaryotes including, by example and not by way of limitation, plants, trypanosomes (Shi et. al., 2000) Drosophila spp. (Kennerdell and Carthew, 2000). Recent experiments have shown that RNAi may also function in higher eukaryotes. For example, it has been shown that RNAi can ablate c-mos in a mouse ooctye and also E-cadherin in a mouse preimplanation embryo (Wianny and Zernicka-Goetz, 2000). This suggests that it may be possible to influence the developmental fate of early embryonic cells.

During mammalian development those cells that form part of the embryo up until the formation of the blastocyst are said to be totipotent (e.g. each cell has the developmental potential to form a complete embryo and all the cells required to support the growth and development of said embryo). During the formation of the blastocyst, the cells that comprise the inner cell mass are said to be pluripotential (e.g. each cell has the developmental potential to form a variety of tissues).

Embryonic stem cells (ES cells, those with pluripotentiality) may be principally derived from two embryonic sources. Cells isolated from the inner cell mass are termed embryonic stem (ES) cells. In the laboratory mouse, similar cells can be derived from the culture of primordial germ cells isolated from the mesenteries or genital ridges of days 8.5-12.5 post coitum embryos. These would ultimately differentiate into germ cells and are referred to as embryonic germ cells (EG cells). Each of these types of pluripotential cell has a similar developmental potential with respect to differentiation into alternate cell types, but possible differences in behaviour (eg with respect to imprinting) have led to these cells to be distinguished from one another.

Typically ES/EG cell cultures have well defined characteristics. These include, but are not limited to;

i) maintenance in culture for at least 20 passages when maintained on fibroblast feeder layers;

ii) produce clusters of cells in culture referred to as embryoid bodies;

iii) ability to differentiate into multiple cell types in monolayer culture;

iv) can form embryo chimeras when mixed with an embryo host;

v) express ES/EG cell specific markers.

Until very recently, in vitro culture of human ES/EG cells was not possible. The first indication that conditions may be determined which could allow the establishment of human ES/EG cells in culture is described in WO96/22362. The application describes cell lines and growth conditions which allow the continuous proliferation of primate ES cells which exhibit a range of characteristics or markers which are associated with stem cells having pluripotent characteristics.

More recently Thomson et al (1998) have published conditions in which human ES cells can be established in culture. The above characteristics shown by primate ES cells are also shown by the human ES cell lines. In addition the human cell lines show high levels of telomerase activity, a characteristic of cells which have the ability to divide continuously in culture in an undifferentiated state. Another group (Reubinoff et. al., 2000) have also reported the derivation of human ES cells from human blastocyts. A third group (Shamblott et. al., 1998) have described EG cell derivation.

A feature of ES/EG cells is that, in the presence of fibroblast feeder layers, they retain the ability to divide in an undifferentiated state for several generations. If the feeder layers are removed then the cells differentiate. The differentiation is often to neurones or muscle cells but the exact mechanism by which this occurs and its control remain unsolved.

In addition to ES/EG cells a number of adult tissues contain cells with stem cell characteristics. Typically these cells, although retaining the ability to differentiate into different cell types, do not have the pluripotential characteristics of ES/EG cells. For example haemopoietic stem cells have the potential to form all the cells of the haemopoietic system (red blood cells, macrophages, basophils, eosinophils etc). All of nerve tissue, skin and muscle retain pools of cells with stem cell potential. Therefore, in addition to the use of embryonic stem cells in developmental biology, there are also adult stem cells which may also have utility with respect to determining the factors which govern cell differentiation. Further recent studies have suggested that some stem cells previously thought to be committed to a single fate, (e.g neurons) may indeed possess considerable pluripotentcy in certain situations. Neural stem cells have recently been shown to chimerise a mouse embryo and form a wide range of non-neural tissue (Clark et. al., 2000).

A further group of cells which have relevance to developmental biology are teratocarcinoma cells (EC cells). These cells form tumours referred to as teratomas and have many features in common with ES/EG cells. The most important of these features is the characteristic of pluripotentiality.

Teratomas contain a wide range of differentiated tissues, and have been known in humans for many hundreds of years. They typically occur as gonadal tumours of both men and women. The gonadal forms of these tumours are generally believed to originate from germ cells, and the extra gonadal forms, which typically have the same range of tissues, are thought to arise from germ cells that have migrated incorrectly during embryogenesis. Teratomas are therefore generally classed as germ cell tumours which encompasses a number of different types of cancer. These include seminoma, embryonal carcinoma, yolk sac carcinoma and choriocarcinoma.

The similar biology of EC cells with ES/EG cells has been exploited to study the developmental fates of cells and to identify cell markers commonly expressed in EC cells and ES/EG cells. For example, and not by way of limitation, the expression of specific cell surface markers SSEA-3 (+), SSEA-4 (+), TRA-1-60 (+), TRA-1-81 (+) (Shevinsky et al 1982; Kannagi et al 1983; Andrews et al 1984a; Thomson et al 1995); alkaline phosphatase (+) (Andrews et. al., 1996); and Oct. 4 (Scholer et. al., 1989; Kraft et. al., 1996; Reubinoff et. al., 2000; Yeom et. al., 1996).

We have accumulated expression studies which identify a number of genes thought to be involved in determining the developmental fate of stem cells, particularly embryonic stem cells. By Northern blotting we have identified the expression of human homologs of two signalling pathways believed to be critical in cell fate determination. Expression of ligands, receptors and downstream components of the Notch and Wingless signalling cascades have been elucidated. Using the model system NTERA2/D1 embryonal carcinoma cells we have recorded changes in the expression of some of these components as the cells differentiate. Baring in mind the role these cascades play in embryonic development throughout the animal kingdom, these changes suggest a significant role for both the wingless and Notch signalling pathways in differentiation of stem cells. Furthermore the activity of some genes are required for differentiation to occur along specific pathways e.g. the myogenic gene MyoD1. Other genes have activity which inhibits cellular differentiation along particular pathways. We envisage regulation of stem cell differentiation to yield a specific cell type could be achieved by:

(i) inhibition of certain genes that normally promote differentiation along particular pathways; therefore promoting differentiation to alternate cell phenotypes;

(ii) inhibition of gene activity that prevents differentiation into particular cell types; and

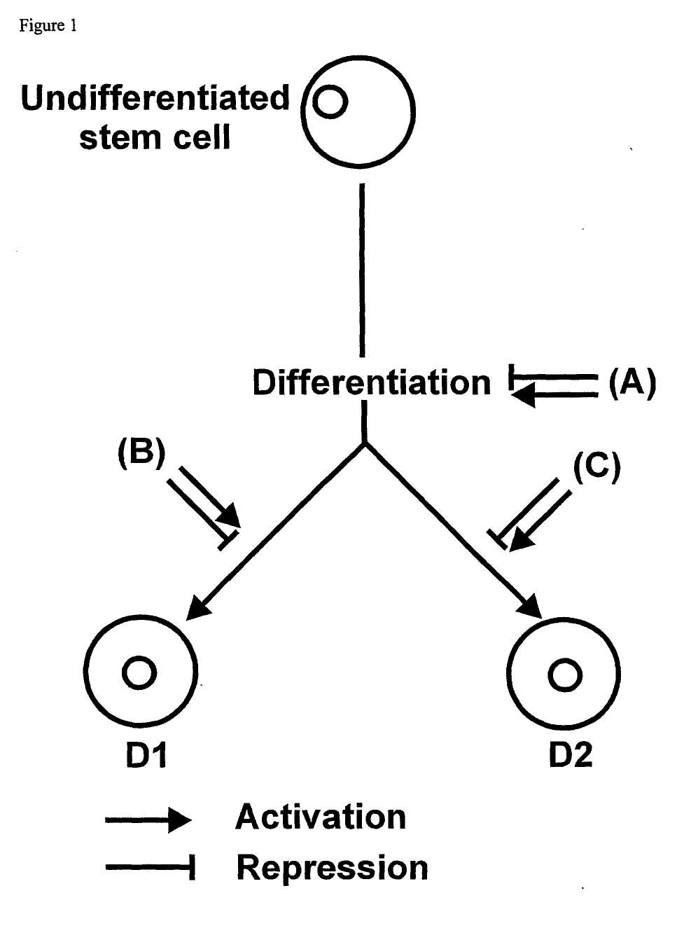

(iii) a combination of (i) and (ii), see FIG. 1

The differentiation of stem cells during embryogenesis, during tissue renewal in the adult and wound repair is under very stringent regulation: aberrations in this regulation underlie the formation of birth defects during development and are thought to underlie cancer formation in adults. Generally, it is envisaged that such stem cells are under both positive and negative regulation which allows a fine degree of control over the process of cell proliferation and cell differentiation: excess proliferation at the expense of cell differentiation can lead to the formation of an expanding mass of tissue—a cancer—whereas express differentiation at the expense of proliferation can lead to the loss of stem cells and production of too little differentiated tissue in the long term, and especially the loss of regenerative potential. Certain genes have already been identified to have a negative role in preventing stem cell differentiation. Such genes, like those of the Notch family, when mutated to acquire activity can inhibit differentiation; such mutant genes act as oncogenes. On the contrary, loss of function of such genes on their inhibition results in stem cell differentiation. We propose to use EC cells has our model cell system to follow the effects of RNAi on cell fate.

According to a first aspect of the invention there is provided a method to modulate the differentiation state of a stem cell comprising:

(i) contacting a stem cell with at least one inhibitory RNA (RNAi) molecule comprising a sequence of a gene, or the effective part thereof, which mediates at least one step in the differentiation of said cell;

(ii) providing conditions conducive to the growth and differentiation of the cell treated in (i) above; and optionally

(iii) maintaining and/or storing the cell in a differentiated state.

The stem cell in (i) above may be a teratocarcinoma cell.

In a preferred method of the invention said conditions are in vitro cell culture conditions.

In a preferred method of the invention said stem cell is selected from: pluripotent stem cells such as an embryonic stem cell or embryonic germ cell; and lineage restricted stem cells such as, but not restricted to; haemopoietic stem cell; muscle stem cell; nerve stem cell; skin dermal sheath stem cell.

It will be apparent that the method can provide stem cells of intermediate commitment. For example, embryonic stem cells could be programmed to differentiate into haemopoietic stems cells with a restricted commitment. Alternatively, differentiated cells or stem cells of intermediate commitment could be reprogrammed to a more pluripotential state from which other differentiated cell lineages can be derived.

In a further preferred method of the invention said stem cell is an embryonic stem cell or embryonic germ cell.

In a yet further preferred method of the invention said gene encodes a cell surface receptor expressed by the stem cell.

In a further preferred method of the invention said cell surface receptor is selected from: human Notch 1(hNotch 1); hNotch 2; hNotch 3; hNotch 4; TLE-1; TLE-2; TLE-3; TLE-4; TCF7; TCF7L1; TCFFL2; TCF3; TCF19; TCF1; mFringe; lFringe; rFringe; sel 1; Numb; Numblike; LNX; FZD1; FZD2; FZD3; FZD4; FZD5; FZD6; FZD7; FZD8; FZD9; FZD10; FRZB.

In an alternative preferred method of the invention said gene encodes a ligand.

Typically, a ligand is a polypeptide which binds to a cognate receptor to induce or inhibit an intracellular or intercellular response. Ligands may be soluble or membrane bound.

In a further alternative preferred method of the invention said ligand is selected from: D11-1; D113; D114; Dlk-1; Jagged 1; Jagged 2; Wnt 1; Wnt 2; Wnt 2b; Wnt 3; Wnt 3a; Wnt5a; Wnt6; Wnt7a; Wnt7b; Wnt8a; Wnt8b; Wnt10b; Wnt11; Wnt14; Wnt15.

Alternatively, said gene is selected from: SFRP1; SFRP2; SFRP4; SFRP5; SK; DKK3; CER1; WIF-1; DVL1; DVL2; DVL3; DVL1L1; mFringe; lFringe; rFringe; sel11; Numb; LNX Oct4; NeuroD1; NeuroD2; NeuroD3; Brachyury; MDFI.

In a further preferred method of the invention of the invention said sequence comprises at least one of the sequences identified in Table 4 which are incorporated by reference.

In a yet further preferred method according to the invention said gene is selected from the group consisting of: DLK1; Oct 4; hNotch 1; hNotch 2; RBPJk; and CIR.

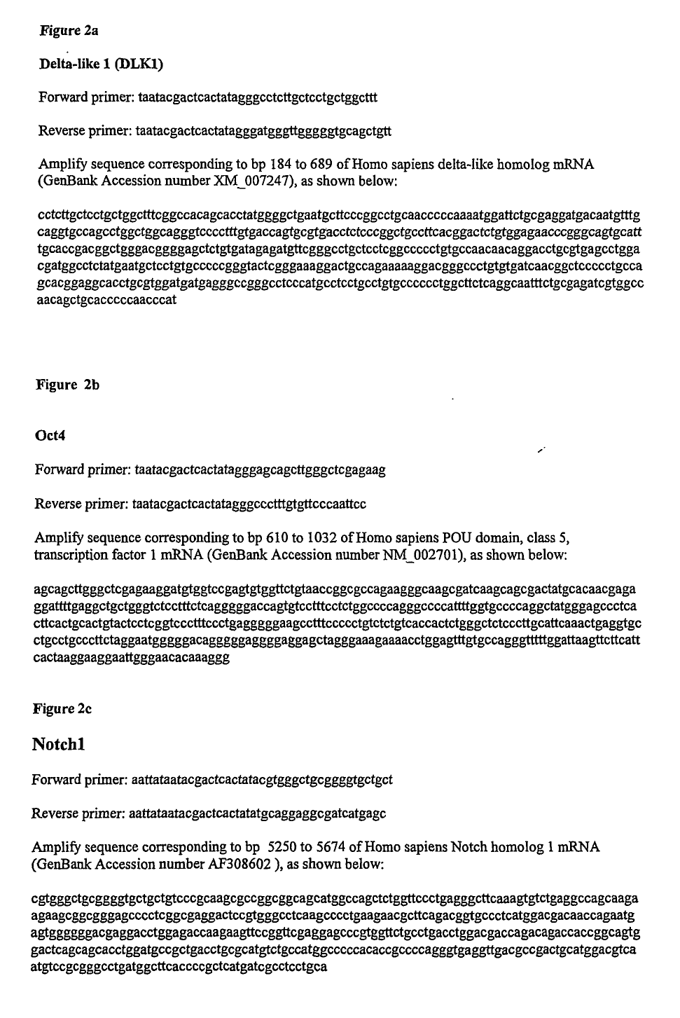

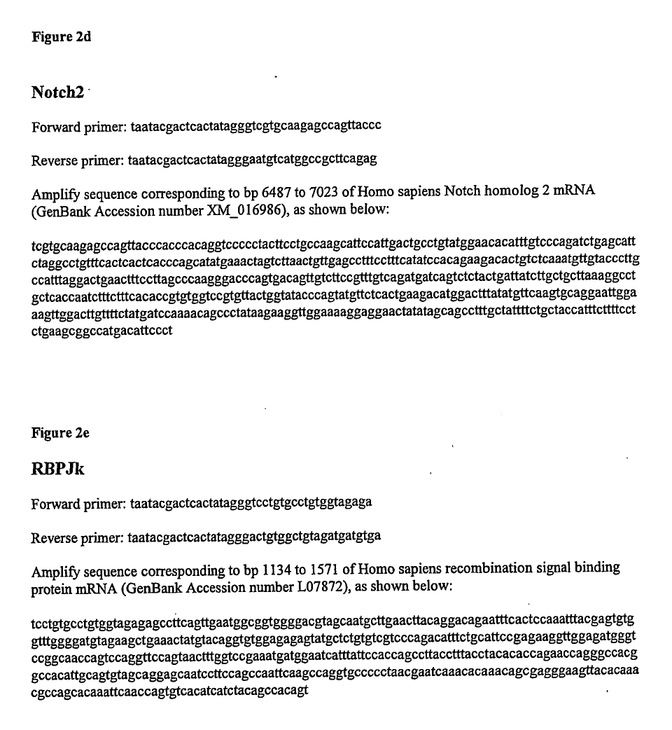

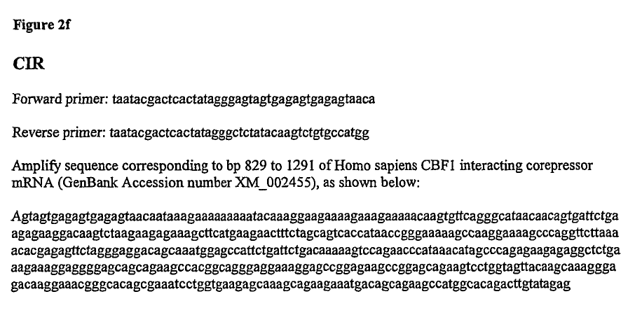

In a further preferred method according to the invention said gene is DLK1. Preferably the DLK1 RNAi molecule is derived from the nucleic acid sequence comprising the sequence presented in FIG. 2a.

In a further preferred method according to the invention said gene is Oct 4. Preferably the Oct 4 RNAi molecule is derived from the nucleic acid sequence comprising the sequence presented in FIG. 2b.

In a further preferred method according to the invention said gene is hNotch 1. Preferably said hNotch 1 RNAi molecule is derived from the nucleic acid sequence comprising the sequence presented in FIG. 2c.

In a further preferred method according to the invention said gene is hNotch 2. Preferably said hNotch 2 RNAi molecule is derived from the nucleic acid sequence comprising the sequence presented in FIG. 2d.

In a further preferred method according to the invention said gene is RBPJk. Preferably said RBPJk RNAi molecule is derived from the nucleic acid sequence comprising the sequence presented in FIG. 2e. RBPJk is also referred to as CBF-1.

In a further preferred method according to the invention said gene is CIR. Preferably said CIR RNAi molecule is derived from the nucleic acid sequence comprising the sequence presented in FIG. 2f.

Many methods have been developed over the last 30 years to facilitate the introduction of nucleic acid into cells which are well known in the art and are applicable to RNAi's.

Methods to introduce nucleic acid into cells typically involve the use of chemical reagents, cationic lipids or physical methods. Chemical methods which facilitate the uptake of DNA by cells include the use of DEAE-Dextran (Vaheri and Pagano Science 175: p434). DEAE-dextran is a negatively charged cation which associates and introduces the nucleic acid into cells. Calcium phosphate is also a commonly used chemical agent which when co-precipitated with nucleic acid introduces the nucleic acid into cells (Graham et al Virology (1973) 52: p456).

The use of cationic lipids (eg liposomes (Felgner (1987) Proc. Natl. Acad. Sci USA, 84:p7413) has become a common method. The cationic head of the lipid associates with the negatively charged nucleic acid backbone to be introduced. The lipid/nucleic acid complex associates with the cell membrane and fuses with the cell to introduce the associated nucleic acid into the cell. Liposome mediated nucleic acid transfer has several advantages over existing methods. For example, cells which are recalcitrant to traditional chemical methods are more easily transfected using liposome mediated transfer.

More recently still, physical methods to introduce nucleic acid have become effective means to reproducibly transfect cells. Direct microinjection is one such method which can deliver nucleic acid directly to the nucleus of a cell (Capecchi (1980) Cell, 22:p479). This allows the analysis of single cell transfectants. So called “biolistic” methods physically shoot nucleic acid into cells and/or organelles using a particle gun (Neumann (1982) EMBO J, 1: p841). Electroporation is arguably the most popular method to transfect nucleic acid. The method involves the use of a high voltage electrical charge to momentarily permeabilise cell membranes making them permeable to macromolecular complexes.

More recently still a method termed immunoporation has become a recognised techinque for the introduction of nucleic acid into cells, see Bildirici et al Nature (2000) 405, p298. The technique involves the use of beads coated with an antibody to a specific receptor. The transfection mixture includes nucleic acid, antibody coated beads and cells expressing a specific cell surface receptor. The coated beads bind the cell surface receptor and when a shear force is applied to the cells the beads are stripped from the cell surface. During bead removal a transient hole is created through which nucleic acid and/or other biological molecules can enter. Transfection efficiency of between 40-50% is achievable depending on the nucleic acid used. In addition the specificity of cell delivery of RNAi's can be enhanced by association or linkage of the RNAi to specific antibodies, ligands or receptors.

According to a further aspect of the invention there is provided an RNAi molecule characterised in that it comprises the coding sequence of at least one gene which mediates at least one step in stem cell differentiation.

In a preferred embodiment said coding sequence is an exon.

Alternatively said RNAi molecule is derived from intronic sequences or the 5′ and/or 3′ non-coding sequences which flank coding/exon sequences of genes which mediate stem cell differentiation.

In a further preferred embodiment of the invention the length of the RNAi molecule is between 100 bp-1000 bp. More preferably still the length of RNAi is selected from 100 bp; 200 bp; 300 bp; 400 bp; 500 bp; 600 bp; 700 bp; 800 bp; 900 bp; or 1000 bp. More preferably still said RNAi is at least 1000 bp.

In an alternative preferred embodiment of the invention the RNAi molecule is between 15 bp and 25 bp, preferably said molecule is 21 bp.

In a further preferred embodiment of the invention said RNAi molecule comprises sequences identified in Table 4 which are incorporated by reference.

In a preferred embodiment of the invention said RNAi molecule is derived from a gene selected from the group consisting of: DLK1; Oct 4; hNotch 1; hNotch 2; RBPJk; and CIR. Preferably said RNAi molecule comprise a nucleic acid sequence selected from the group consisting of the nucleic acid sequences presented in FIGS. 2a-2f.

In yet a further preferred embodiment of the invention said RNAi molecules comprise modified ribonucleotide bases.

It will be apparent to one skilled in the art that the inclusion of modified bases, as well as the naturally occuring bases cytosine, uracil, adenosine and guanosine, may confer advantageous properties on RNAi molecules containing said modified bases. For example, modified bases may increase the stability of the RNAi molecule thereby reducing the amount required to produce a desired effect.

According to a further aspect of the invention there is provided an isolated DNA molecule comprising a sequence of a gene which mediates at least one step in stem cell differentiation as represented by the DNA accession numbers identified in Table 4 characterised in that said DNA is operably linked to at least one further DNA molecule capable of promoting transcription (“a promoter”) of said DNA linked thereto.

In a preferred embodiment of the invention said gene is selected from the group consisting of: DLK1; Oct 4; hNotch 1; hNotch 2; RBPJk; and CIR. Preferably said DNA comprises a sequence selected from the group consisting of the sequences as represented in FIGS. 2a-2f.

In a further preferred embodiment of the invention said gene is provided with at least two promoters characterised in that said promoters are oriented such that both DNA strands comprising said DNA molecule are transcribed into RNA.

It will be apparent to one skilled in the art that the synthesis of RNA molecules which form RNAi can be achieved by providing vectors which include target genes, or fragments of target genes, operably linked to promoter sequences. Typically, promoter sequences are phage RNA polymerase promoters (eg T7, T3, SP6). Advantageously vectors are provided with with multiple cloning sites into which genes or gene fragments can be subcloned. Typically, vectors are engineered so that phage promoters flank multiple cloning sites containing the gene of interest. Phage promoters are oriented such that one promoter synthesises sense RNA and another phage promoter, antisense RNA. Thus, the synthesis of RNAi is facilitated.

Alternatively target genes or fragments of target genes can be fused directly to phage promoters by creating chimeric promoter/gene fusions via oligo-synthesising technology. Constructs thus created can be easily amplified by polymerase chain reaction to provide templates for the manufacture of RNA molecules comprising RNAi.

According to a further aspect of the invention there is provided a vector including a DNA molecule according to the invention.

According to a farther aspect of the invention there is provided a method to manufacture RNAi molecules comprising:

(i) providing DNA molecule or vector according to the invention;

(ii) providing reagents and conditions which allow the synthesis of each RNA strand comprising said RNAi molecule; and

(iii) providing conditions which allow each RNA strand to associate over at least part of their length, or at least that part corresponding to the nucleic acid sequence encoding said stem cell gene which mediates stem cell differentiation.

Preferably said gene, or gene fragment is selected from those genes represented in table 4.

In vitro transcription of RNA is an established methodology. Kits are commercially available which provide vectors, ribonucleoside triphosphates, buffers, Rnase inhibitors, RNA polymersases (eg phage T7, T3, SP6) which facilitate the production of RNA.

According to a further aspect of the invention there is provided an in vivo method to promote the differentiation of stem cells comprising administering to an animal an effective amount of RNAi according to the invention sufficient to effect differentiation of a target stem cell. Preferably said method promotes differentiation in vivo of endogenous stem cells to repair tissue damage in situ.

It will be apparent to one skilled in the art that RNAi relies on homology between the target gene RNA and the RNAi molecule. This confers a significant degree of specificity to the RNAi molecule in targeting stem cells. For example, haemopoietic stem cells are found in bone marrow and RNAi molecules may be administered to an animal by direct injection into bone marrow tissue.

RNAi molecules may be encapsulated in liposomes to provide protection from an animals immune system and/or nucleases present in an animals serum.

Liposomes are lipid based vesicles which encapsulate a selected therapeutic agent which is then introduced into a patient. Typically, the liposome is manufactured either from pure phospholipid or a mixture of phospholipid and phosphoglyceride. Typically liposomes can be manufactured with diameters of less than 200 nm, this enables them to be intravenously injected and able to pass through the pulmonary capillary bed. Furthermore the biochemical nature of liposomes confers permeability across blood vessel membranes to gain access to selected tissues. Liposomes do have a relatively short half-life. So called STEALTH^{R} liposomes have been developed which comprise liposomes coated in polyethylene glycol (PEG). The PEG treated liposomes have a significantly increased half-life when administered intravenously to a patient. In addition STEALTH^{R} liposomes show reduced uptake in the reticuloendothelial system and enhanced accumulation selected tissues. In addition, so called immuno-liposomes have been develop which combine lipid based vesicles with an antibody or antibodies, to increase the specificity of the delivery of the RNAi molecule to a selected cell/tissue.

The use of liposomes as delivery means is described in U.S. Pat. No. 5,580,575 and U.S. Pat. No. 5,542,935.

It will be apparent to one skilled in the art that the RNAi molecules can be provided in the form of an oral or nasal spray, an aerosol, suspension, emulsion, and/or eye drop fluid. Alternatively the RNAi molecules may be provided in tablet form. Alternative delivery means include inhalers or nebulisers.

According to a yet further aspect of the invention there is provided a therapeutic composition comprising at least one RNAi molecule according to the invention.

Preferably said RNAi molecule is for use in the manufacture of a medicament for use in promoting the differentiation of stem cells to provide differentiated cells/tissues to treat diseases where cell/tissues are destroyed by said disease. Typically this includes pernicious anemia; stroke, neurodegenerative diseases such as Parkinson's disease, Alzhiemer's disease; coronary heart disease; cirrhosis; diabetes. It will also be apparent that differentiated stem cells may be used to replace nerves damaged as a consequence of (eg replacement of spinal cord tissue).

In a further preferred embodiment of the invention said therapeutic composition further comprises a diluent, carrier or excipient.

According to a further aspect of the invention there is provided a therapeutic cell composition comprising a differentiated cell produced by introduction of a RNAi molecule or composition according to the invention.

According to a further aspect of the invention there is provided a cell obtainable by the method according to the invention.

In a preferred embodiment of the invention said cell is selected from the group consisting of: a nerve cell; a mesenchymal cell; a muscle cell (cardiomyocyte); a liver cell; a kidney cell; a blood cell (eg erythrocyte, CD4+ lymphocyte, CD8+ lymphocyte; panceatic β cell; epithelial cell (eg lung, gastric,); and a endothelial cell.

According to a further aspect of the invention there is provided a cell culture obtainable by the method according to the invention.

According to a yet further aspect of the invention there is provided at least one organ comprising at least one cell according to the invention.

An embodiment of the invention will now be described by example only and with reference to the following figures and tables wherein:

Table 1 represents a selection of antibodies used to monitor stem cell differentiation;

Table 2 represents nucleic acid probes used to assess mRNA markers of stem differentiation;

Table 3 represents protein markers of stem cell differentiation;

Table 4 represents specific primers used to generate RNAi for gene specific inhibition and gene sequences with DNA database accession numbers;

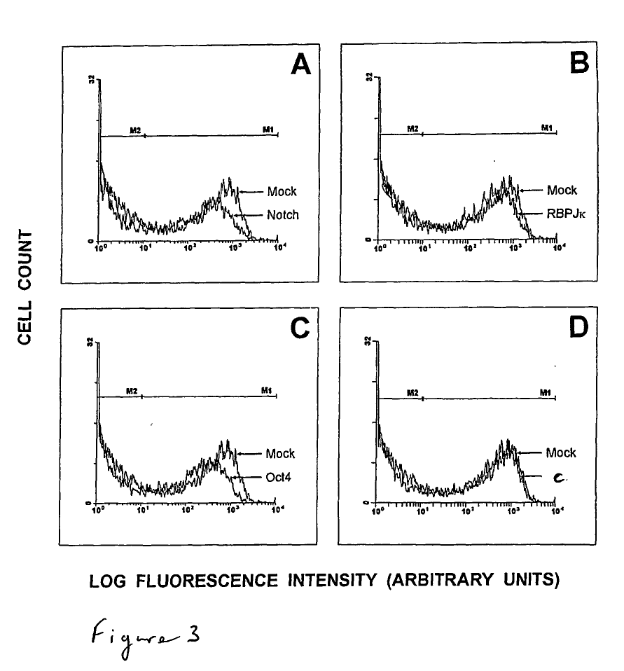

Table 5 represents a summary of FACS data presented in FIG. 3;

FIG. 1 illustrates stem cell differentiation is controlled by positive and negative regulators (A). The specific cell phenotypes that are derived are a direct result of positive and negative regulators which activate or suppress particular differentiation events. RNAi can be used to control both the initial differentiation of stem cells (A) and the ultimate fate of the differentiated cells D1 and D2 by repression of positive activators which would normally promote a particular cell fate;

FIG. 2a represents the forward and reverse primers used to amplify delta-like 1 (DLK1) and the amplified sequence; FIG. 2b represents the forward and reverse primers used to amplify Oct 4 and the amplified sequence; FIG. 2c represents the forward and reverse primers used to amplify Notch 1 and the amplified sequence; FIG. 2d represents the forward and reverse primers used to amplify Notch 2 and the amplified sequence; FIG. 2e represents the forward and reverse primers used to amplify RBPJK and the amplified sequence; and FIG. 2f represents the forward and reverse primers used to amplify CIR and the amplified sequence;

FIG. 3 represents a FACS scan of monitoring the expression of SSEA3 by NTERA2cl D1 human EC cells following RNAi to Notch (A), RBPJk(B), Oct 4 (C) and control RNAi (D). Flow cytofluorimetric analysis of SSEA3 expression by NTERA2 cl.D1 human EC cells, 4 days following transfection with RNAi directed to a) Notch1 and Notch2; b) RBPJk; c) Oct4; d) control RNAi. Each panel shows two histograms of cell number against log fluorescence intensity (arbitrary units), after staining cells with monoclonal antibody MC631 (anti SSEA3) followed by FITC labelled goat anti-mouse IgM. In each panel, one histogram was derived from ‘mock’ transfected cells that had been treated with all relevant reagents except RNAi; the second histogram in each panel was derived from cells treated with RNAi directed to the set of genes as described above. Note that the cells exhibit a bimodal histogram in all cases representing SSEA3+ and SSEA3− populations (regions marked M1 and M2 respectively). Note that following treatment with RNAi to Notch1 and Notch2 (Panel A) and Oct4 (Panel c), there was a marked downward shift in the fluorescence intensity of the SSEA3+ population, denoting evidence of stem cell differentiation. A smaller shift, also downwards, was evident in cells treated with RBPJk (Panel B). Such results would be anticipated if these gene products play a role in maintenance of an undifferentiated EC cell phenotype, and if treatment with RNAi directed to the corresponding mRNA results in down regulation of these key regulatory proteins. By contrast, treatment with control RNAi (Panel D) did not result in any down regulation of SSEA3. Expression of SSEA3 appears to be a very sensitive marker of an undifferentiated EC stem cell phenotype and is one of the most rapid markers to disappear upon differentiation (Fenderson et al 1987; Andrews et al 1996). Likewise SSEA3 is expressed by human ES cells (Thomson et al 1998) and also disappears rapidly upon their differentiation (P W Andrews and J S Draper, unpublished results);

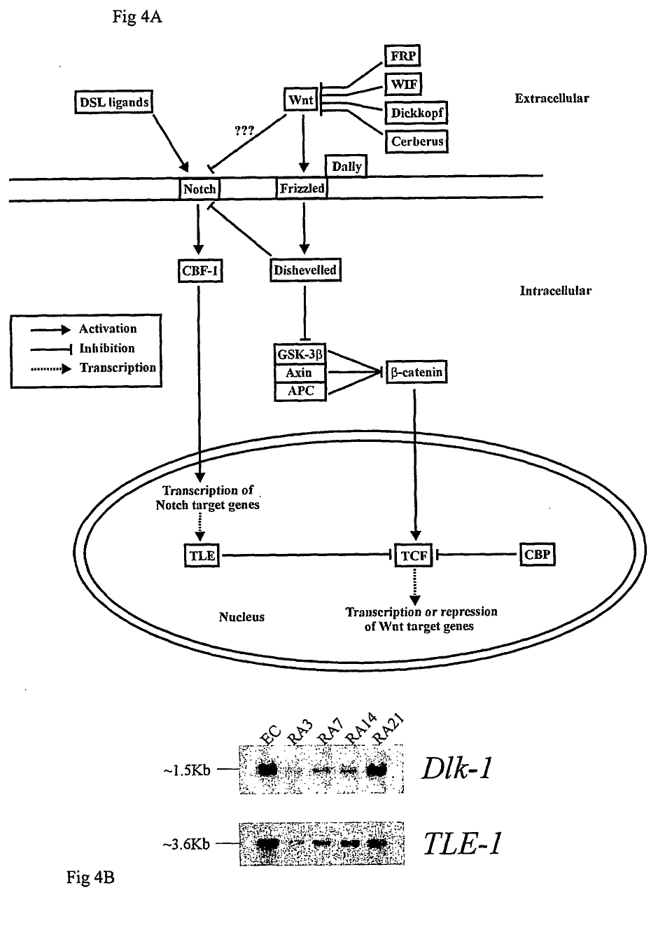

FIG. 4 represents (A) a schematic diagram illustrating the Notch and Wnt signalling pathways. The Notch and Wnt signaling pathways are shown. Ligands of the Delta/Serrate/Lag (DSL) family bind Notch receptors, leading to activation of Suppressor of Hairless (Su-H)/CBF1/RBPJk and enhanced transcription of target genes. (B) a northern blot analysis of the expression of the DLS ligand Dlk and the Notch target gene TLE1 in NTERA2 EC cells. TLE1 was identified as a target gene of the Notch pathway in NTERA2 EC cells. TLE1 shows a pattern of expression highly similar to that of the DSL ligand, Dlk1, during retinoic acid-induced differentiation. At 3 days following RA treatment (RA3), both genes are substantially downregulated. At subsequent time points, a progressive recovery in expression is seen, through to 21 days after RA treatment (RA21). The downregulation of TLE1 indicates that the cells have entered a differentiation pathway. (C) RT PCR analysis of TLE1 and HASH1 in RNAi treated ES cells. RT-PCR was performed for TLE1 and HASH1 3 days after dsRNA treatment. Lane 1: water; lane 2: untreated ES cells; lane 3: mock transfection; lane 4: Notch 1& 2 dsRNA; lane 5: DlkI dsRNA; lane 6: RBPJk dsRNA; lane 7: CIR dsRNA; lane 8: Oct4 dsRNA; lane 9: control dsRNA. Note the specific reduction of TLE1 expression in lanes 5 and 6, corresponding to samples in which components of the Notch signaling pathway have been targeted by dsRNA. Also note the appearance of HASH1 in lane 5. These data indicate that the cells are embarking on a program of neural differentiation (de la Pompa et al, Conservation of the Notch signalling pathway in mammalian neurogenesis. Development 124, 1139-1148 (1997). The failure of Notch1&2 dsRNA to induce a similar effect is due to functional redundancy of the receptor system, or a high abundance of receptor in relation to other pathway components.

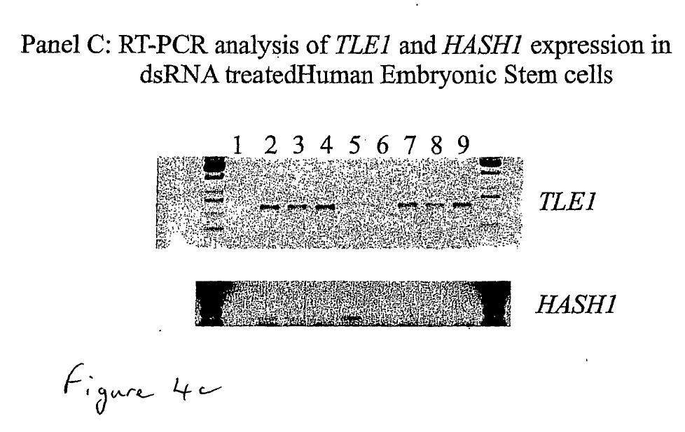

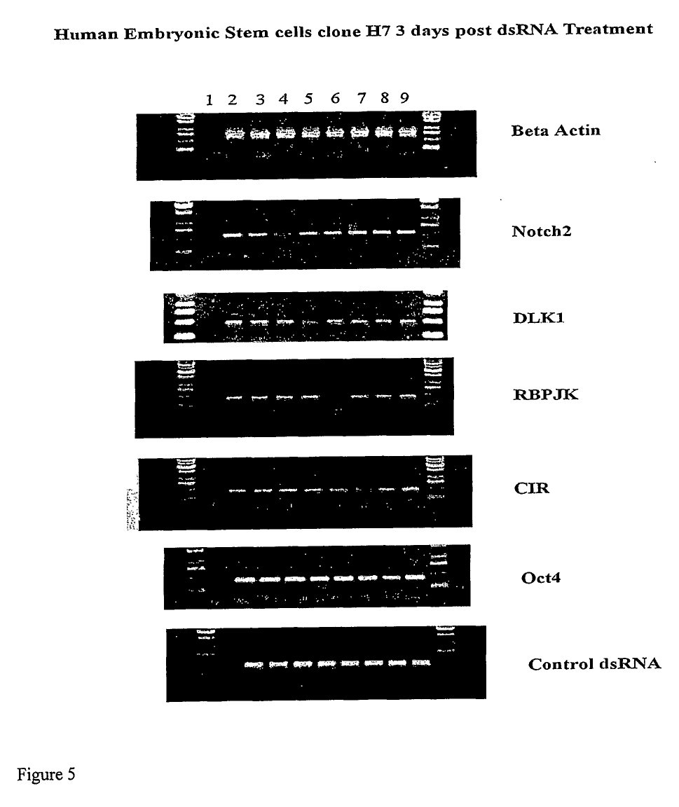

FIG. 5 represents RNAi of human ES cells using RNAi molecules derived from different genes involved in stem cell differentiation using RT PCR to monitor steady-state levels of mRNA. RT-PCR analysis of targeted transcript abundance in human embryonic stem cells 3 days after dsRNA treatment. Lane 1: water; lane 2: untreated ES cells; lane 3: mock transfection; lane 4: Notch 1&2 dsRNA; lane 5: Dlk1 dsRNA; lane 6: RBPJk (CBF]) dsRNA; lane 7: CIR dsRNA; lane 8: Oct4 dsRNA; lane 9: control dsRNA. Note that specific reduction in targeted transcript abundance persists for at least 3 days after dsRNA treatment. The effect is especially prominent in cells treated with the Notch 1&2, RBPJk (CBF1) and Oct4 dsRNAs. Beta Actin PCR was used as a template loading control for PCR.

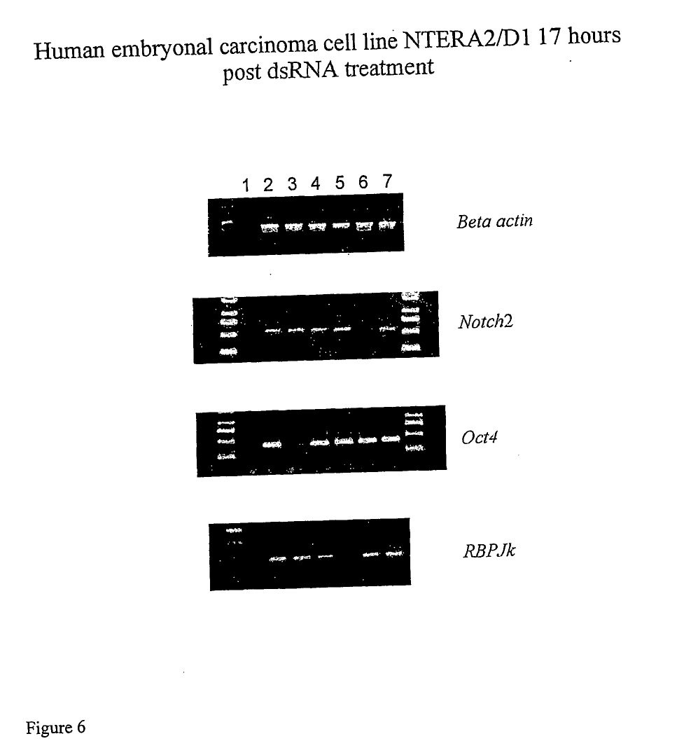

FIG. 6 represents RNAi of NTERA2/D1 using RNAi molecules derived from different genes involved in stem cell differentiation using RT PCR to monitor steady-state levels of mRNA. RT-PCR analysis of targeted transcript abundance in the human embryonal carcinoma cell line, NTERA2, 17 hours after dsRNA treatment. Lane 1: water; lane 2: untreated EC cells; lane 3: Oct4 dsRNA; lane 4:control dsRNA; lane 5: RBPJk dsRNA; lane 6: Notch 1&2 dsRNA; lane 7: mock transfection. Note the specific and substantial reduction of targeted transcript abundance. Beta Actin PCR was used as a template loading control.

### MATERIALS AND METHODS

Cell Culture

NTERA2 and 2102Ep human EC cell lines were maintained at high cell density as previously described (Andrews et al 1982, 1984b), in DMEM (high glucose formulation) (DMEM)(GIBCO BRL), supplemented with 10% v/v bovine foetal calf serum (GIBCO BRL), under a humidified atmosphere with 10% CO₂ in air.

Double Stranded RNA Synthesis

PCR primers were designed against the mRNA sequence of interest to give a product size of around 500 bp. At the 5′ end of each primer was added a T7 RNA polymerase promoter, comprising one or other of the following sequences: TAATACGACTCACTATAGGG; AATTATAATACGACTCACTATA. PCR was performed using these primers on an appropriate cDNA source (e.g. derived from the cell type to be targeted) and the product cloned and sequenced to confirm its identity. Using the sequenced clone as a template, further PCRs were performed as required to generate template DNA for RNA synthesis. In each case, a quantity of the PCR was electrophoresed through agarose to verify product size and abundance, whilst the remainder was purified by alkaline phenol/chloroform extraction. RNA was synthesized using the Megascript kit (Ambion Inc.) according to the manufacturer's protocol and acid phenol/chloroform extracted. The simultaneous synthesis of complementary strands of RNA in a single reaction circumvents the requirement for an annealing step. However, the quality and duplexing of the synthesized RNA was confirmed by agarose gel electrophoresis, with the desired products migrating as expected for double stranded DNA of the same length.

Treatment of Human Cells with dsRNA to Produce RNAi

The following method describes RNAi of cells cultured in 6 well plates. Volumes and cell numbers should be scaled appropriately for larger or smaller culture vessels. Cells were seeded at 500,000 per well on the day prior to treatment and grown in their normal medium. For each well to be treated, 9.5 μg of the double stranded RNA of interest was diluted in 300 μl of 150 mM NaCl. 21 μl of ExGen 500 (MBI Fermentas) was added to the diluted RNA solution and mixed by vortexing. The dsRNA/ExGen 500 mixture was incubated at room temperature for 10 minutes. 3ml of fresh cell growth medium was then added, producing the RNAi treatment medium. Growth medium was aspirated from the culture vessel and replaced with 3ml of RNAi treatment medium per well. Culture vessels were then centrifuged at 280 g for 5 minutes and returned to the incubator. After 12-18 hrs, RNAi treatment medium was replaced with normal growth medium and the cells maintained as required.

Oct 4 RNAi Production

PCR primers were designed against the Oct 4 mRNA sequence of interest to give a product size of around 500 bp. At the 5′ end of each primer was added a T7 RNA polymerase promoter, comprising the following sequence: taatacgactcactataggg. PCR was performed using these primers on an appropriate cDNA source (e.g. derived from the cell type to be targeted) and the product cloned and sequenced to confirm its identity. Using the sequenced clone as a template, further PCRs were performed as required to generate template Oct 4 DNA for RNA synthesis. In each case, a quantity of the PCR was electrophoresed through agarose to verify product size and abundance, whilst the remainder was purified by alkaline phenol/chloroform extraction. RNA was synthesized using the Megascript kit (Ambion Inc.) according to the manufacturer's protocol and acid phenol/chloroform extracted. The simultaneous synthesis of complementary strands of RNA in a single reaction circumvents the requirement for an annealing step. However, the quality and duplexing of the synthesized RNA was confirmed by agarose gel electrophoresis, with the desired products migrating as expected for double stranded DNA of the same length.

Treatment of Human EC Cells with Oct 4 dsRNA to Produce RNAi

The following method describes Oct 4 RNAi of cells cultured in 6 well plates. Volumes and cell numbers should be scaled appropriately for larger or smaller culture vessels.

Cells were seeded at 500,000 per well on the day prior to treatment and grown in their normal medium. On the day of treatment, a 15 ul aliquot of Lipofectin (Gibco BRL) was added to 100 μl of Optimem (Gibco BRL) for each well to be treated. Concurrently, 6 ug of Oct 4 dsRNA was added to 300 ul of Optimem for each well to be treated. The Lipofectin-Optimem and dsRNA-Optimem solutions were incubated at room temperature for 40 minutes, then mixed to produce RNAi treatment medium with a total volume of around 415 ul for each well. The treatment medium was incubated at room temperature for 10 minutes prior to use. During this time, growth medium was aspirated from the cells and each well washed with 3 ml of PBS. The PBS wash was then replaced with RNAi treatment medium, supplemented with a further 0.5 ml of Optimem per well. Culture vessels were returned to the incubator for 6.5 hours, after which the treatment medium was aspirated and replaced with normal growth medium. Target mRNA inhibition was assayed 3 days after treatment by PCR.

RNAi Introduction to Cell Lines

Human EC stem cells were seeded at 2×10⁵ cells/well of a 6 well plate in 3 cm³ of Dulbecco's modified Eagles medium and allowed to settle for 3 hrs. 6 μg RNAi was added to the medium and the cells were agitated for 30 mins at room temperature.

Foetal calf serum (GIBO BRL) was added to the medium to a concentration of 10% and the cells were grown on.

Total RNA Production

Growing cultures of cells were aspirated to remove the DME and foetal calf serum. Trace amounts of foetal calf serum was removed by washing in Phosphate-buffered saline. Fresh PBS was added to the cells and the cells were dislodged from the culture vessel using acid washed glass beads. The resulting cell suspension was centrifuged at 300×g. The pellets had the PBS aspirated from them. Tri reagent (Sigma, USA) was added at 1 ml per 10⁷ cells and allowed to stand for 10 mins at room temperature. The lysate from this reaction was centrifuged at 12000×g for 15 minutes at 4° C. The resulting aqueous phase was transferred to a fresh vessel and 0.5 ml of isopropanol/ml of trizol was added to precipitate the RNA. The RNA was pelleted by centrifugation at 12000×g for 10 mins at 4° C. The supernatant was removed and the pellet washed in 70% ethanol. The washed RNA was dissolved in DEPC treated double-distilled water.

Analysis of the Differentiation of EC Stem Cells Induced by Exposure to RNAi

Following exposure to RNAi corresponding to specific key regulatory genes, the subsequent differentiation of the EC cells was monitored in a variety of ways. One approach was to monitor the disappearance of typical markers of the stem cell phenotype; the other was to monitor the appearance of markers pertinent to the specific lineages induced. The relevant markers included surface antigens, mRNA species and specific proteins.

Analysis of Transfectants by Antibody Staining and FACS

Cells were treated with trypsin (0.25% v/v) for 5 mins to disaggregate the cells; they were washed and re-suspended to 2×10⁵ cells/ml. This cell suspension was incubated with 50 μl of primary antibody in a 96 well plate on a rotary shaker for 1 hour at 4° C. Supernatant from a myeloma cell line P3X63Ag8, was used as a negative control. The 96 well plate was centrifuged at 100 rpm for 3 minutes. The plate was washed 3 times with PBS containing 5% foetal calf serum to remove unbound antibody. Cell were then incubated with 50 μl of an appropriate FITC-conjugated secondary antibody at 4° C. for 1 hour. Cells were washed 3 times in PBS +5% foetal calf serum and analysed using an EPICS elite ESP flow cytometer (Coulter eletronics, U.K).(Andrews et. al., 1982)

Northern Blot Analysis of RNA

RNA separation relies on the generally the same principles as standard DNA but with some concessions to the tendancy of RNA to hybridise with itself or other RNA molecules. Formaldehyde is used in the gel matrix to react with the amine groups of the RNA and form Schiff bases. Purified RNA is run out using standard agarose gel electrophresis. For most RNA a 1% agarose gel is sufficiant. The agarose is made in 1×MOPS buffer and supplemeted with 0.66M formaldehyde. Dryed down RNA samples are reconstituted and denatured in RNA loading buffer and loaded into the gel. Gels are run out for apprx. 3 hrs (until the dye front is ¾ of the way down the gel).

The major problem with obtaining clean blotting using RNA is the presence of formaldehyde. The run out gel was soaked in distilled water for 20 mins with 4 changes, to remove the formaldehyde from the matrix. The transfer assembly was assembled in exactly the same fashion as for DNA (Southern) blotting. The transfer buffer used however was 10×SSPE. Gels were transfered overnight. The membrane was soaked in 2×SSPE to remove any agarose from the transfer assembly and the RNA was fixed to the memebrane. Fixation was acheived using short-wave (254 nM) UV light. The fixed membrane was baked for 1-2 hrs to drive off any residual formaldehyde.

Hybridisation was acheived in aqueous phase with formamide to lower the hybridisation temperatures for a given probe. RNA blots were prehybridised for 2-4 hrs in northern prehybridisation soloution. Labelled DNA probes were denatured at 95° C. for 5 mins and added to the blots. All hybridisation steps were carried out in rolling bottles in incubation ovens. Probes were hybridised overnight for at least 16 hrs in the prehybridisation soloution. A standard set of wash soloutions were used. Stringency of washing was acheived by the use of lower salt containing wash buffers. The following wash procedure is outlined as follows

| | | |
|---|---|---|
| 2X SSPE | 15 mins | room temp |
| 2X SSPE | 15 mins | room temp |
| 2X SSPE/0.1% SDS | 45 mins | 65° C. |
| 2X SSPE/0.1% SDS | 45 mins | 65° C. |
| 0.1X SSPE | 15 mins | room temp |

Preparation of Radiolabelled DNA Probes

The method of Feinberg and Vogelstein (Feinberg and Vogelstein, 1983) was used to radioactively label DNA. Briefly, the protocol uses random sequence hexanucleotides to prime DNA synthesis at numerous sites on a denatured DNA template using the Klenow DNA polymerase I fragment. Pre-formed kits were used to aid consistency. 5-100 ng DNA fragment (obtained from gel purifcation of PCR or restriction digests) was made up in water,denatured for 5 mins at 95° C. with the random hexamers. The mixture was quench cooled on ice and the following were added,

5 μl[α-32P] DATP 3000 Ci/mmol

1 μl of Klenow DNA polymerase (4U)

The reaction was then incubated at 37° C. for 1 hr. Unincorporated nucleotide were removed with spin columns (Nucleon Biosciences).

Production of cDNA

The enzymatic conversion of RNA into single stranded cDNA was achieved using the 3′ to 5′ polymerase activity of recombinant Moloney-Murine Leukemia Virus (M-MLV) reverse transcriptase primed with oligo (dT) and (dN) primers. For Reverse Transcription-Polymerase Chain Reaction, single stranded cDNA was used. cDNA was synthesised from 1 μg poly (A)+ RNA or total RNA was incubated with the following

| | |
|---|---|
| 1.0 μM | oligo(dT) primer for total RNA or random hexcamers for mRNA |
| 0.5 mM | 10 mM dNTP mix |
| 1 U/μl | RNAse inhibitor (Promega) |
| 1.0 U/μl | M-MLV reverse transcriptase in manufacturers supplied buffer |
| | (Promega) |

The reaction was incubated for 2-3 hours at 42° C.

Fluorescent Automated Sequencing

To check the specificity of the PCR primers used to generate the template used in RNAi production automatic sequencing was carried out using the prism fluorescently labelled chain terminator sequencing kit (Perkin-Elmer) (Prober et al 1987). A suitable amount of template (200 ng plasmid, 100 ng PCR product), 10 μM sequencing primer (typically a 20 mer with 50% G-C content) were added to 8 μl of prism pre-mix and the total reaction volume made up to 20 μl. 24 cycles of PCR (94° C. for 10 seconds, 50° C. for 10 seconds, 60° C. for 4 minutes). Following thermal cycling, products were precipitated by the addition of 2 μl of 3M sodium acetate and 50 μl of 100 % ethanol. DNA was pelleted in an Eppendorf microcentrifuge at 13000 rpm, washed once in 70% ethanol and vacuum dried. Samples were analysed by the in-house sequencing Service (Krebs Institute). Dried down samples were resuspended in 4 μl of formamide loading buffer, denatured and loaded onto a ABI 373 automatic sequencer. Raw sequence was collected and analysed using the ABI prism software and the results were supplied in the form of analysed histogram traces.

Detection of Specific Protein Targets by SDS-PAGE and Western Blotting

To obtain cell lysates monolayers of cells were rinsed 3 times with ice-cold PBS supplemented with 2 mM CaCl₂. Cells were incubated with 1 ml/75 cm² flask lysis buffer (1% v/v NP40, 1% v/v DOC, 0.1 mM PMSF in PBS) for 15 min at 4° C. Cell lysates were transferred to eppendorf tubes and passed through a 21 gauge needle to shear the DNA. This was followed by freeze thawing and subsequent centrifugation (30 min, 4° C., 15000 g) to remove insoluble material. Protein concentrations of the supernatants were determined using a commercial protein assay (Biorad) and were adjusted to 1.3 mg/ml. Samples were prepared for SDS-PAGE by adding 4 times Laemmli electrophoresis sample buffer and boiling for 5 min. After electrophoresis with 16 μg of protein on a 10% polyacrylamide gel (Laemmli, 1970) the proteins were transferred to nitro-cellulose membrane with a pore size of 0.45 μm. The blots were washed with PBS and 0.05% Tween (PBS-T). Blocking of the blots occurred in 5% milk powder in PBS-T (60 min, at RT). Blots were incubated with the appropriate primary antibody. Horseradish peroxidase labelled secondary antibody was used to visualise antibody binding by ECL (Amersham, Bucks., UK). Materials used for SDS-PAGE and western blotting were obtained from Biorad (California, USA) unless stated otherwise.

TABLE 1
Antibodies used to detect stem cell differentiation

| | | | Cell | | |
|---|---|---|---|---|---|
| | | | phenotype | Changes on | |
| Antibody | Class | Species | detected | Differentiation | Reference |
| TRA-1- | IgM | Mouse | Human EC, | ↓ | Andrews et. |
| 60 | | | ES cells. | differentiation | al., 1984a |
| TRA-l- | IgM | Mouse | Human EC, | ↓ | Andrews et. |
| 81 | | | ES cells. | differentiation | al., 1984a |
| SSEA3 | IgM | Rat | Human EC, | ↓ | Shevinskyet |
| | | | ES cells. | differentiation | et al 1982, |
| | | | | | Fenderson |
| | | | | | et al 1987 |
| SSEA4 | IgG | Mouse | Human EC, | ↓ | Kannagi et |
| | | | ES cells. | differentiation | al 1983 |
| | | | | | Fenderson |
| | | | | | et al 1987 |
| A2B5 | IgM | Mouse | | ↑ | Fenderson |
| | | | | differentiation | et al 1987 |
| ME311 | IgG | Mouse | | ↑ | Fenderson |
| | | | | differentiation | et al 1987 |
| VIN-IS- | IgM | Mouse | | ↑ | Andrews |
| 56 | | | | differentiation | et al 1990 |
| VIN-IS- | IgG | Mouse | | ↑ | Andrews |
| 53 | | | | differentiation | et al 1990 |

TABLE 2
Probes used to assess mRNA markers of differentiation

| Gene | Cell Type |
|---|---|
| Synaptophysin | Neuron |
| NeuroD1 | Neuron |
| MyoD1 | Muscle |
| Collagens | Cartlidge |
| Alpha-actin | Skeletal muscle |
| Smooth-muscle actin | Smooth muscle |

TABLE 3
Protein markers of differentiation, detected by
Western Blot and/or immununofluorescence.
The following antibodies were detected by the
appropriate commercially available antibodies

| Cell Type | Antigen |
|---|---|
| Neurons | Neurofilaments |
| Glial cells | GFAP |
| Epithelial cells | Cytokeratins |
| Mesenchymal cells | Vimentin |
| Muscle | Desmin |
| Muscle | Tissue specific actins |
| Connective tissue cells | Collagens |

TABLE 4
Specific Primers used to generate dsRNA for gene specific inhibition
All sequences written 5′ to 3′

| Gene | Accession | | |
|---|---|---|---|
| Name | number | PCR primer Sequences | Position |
| Notch | | | |
| Pathway | | | |
| Ligands: | | | |
| Dll-1 | AF003522 | | |
| Dll3 | NM_016941 | | |
| Dll4 | NM_019454 | | |
| Dlk-1 | NM_003836 | taatacgactcactatagggcctcttgctcct | |
| | | gctggcttt | |
| | | taatacgactcactatagggatgggt | |
| | | tgggggtgcagctgtt | |
| Receptors: | | | |
| Notch1 | M73980 | gcggccgcctttgtggttctgttc | 5224-5726 |
| | | gccggcgcgtcctcctcttcc | |
| Notch2 | Inhouse | gccagaatgatgctacctgt | |
| | sequence | tagagcagcaccaatggaac | |
| Notch3 | U97669 | aagttacccccaagaggcaagtgtt | 7013-7348 |
| | | aaggaaatgagaggccagaagga | |
| | | ga | |
| Notch4 | U95299 | ggctgcccctcccactctcg | 3727-4132 |
| | | cagcccgggccccaggatag | |
| Down- | | | |
| stream: | | | |
| TLE-1 | NM_005077 | | |
| TLE-2 | M99436 | | |
| TLE-3 | M99438 | | |
| TLE-4 | M99439 | | |
| TCF7 | NM_003202 | | |
| TCFFL2 | Y11306 | | |
| TCF3 | M31523 | | |
| TCF19 | NM_007109 | | |
| TCF1 | NM_000545 | | |
| mfringe | NM_002405 | | |
| lfringe | U94354 | | |
| rFringe | AF108139 | | |
| Se11 | AF157516 | | |
| Numb | NM_003744 | | |
| LNX | NM_010727 | | |
| Wingless | | | |
| Pathway | | | |
| Ligands | | | |
| Wnt1 | NM_005430 | | |
| Wnt2 | NM_003391 | | |
| Wnt2B | NM_004185 | tgagtggttcctgtactctg | 1159-1503 |
| | | actcacactgggtaacacgg | |
| Wnt5A | L20861 | | |
| Wnt6 | AF079522 | | |
| Wnt7A | NM_004625 | | |
| Wnt8B | NM_003393 | | |
| Wnt10B | NM_003394 | | |
| Wnt11 | NM_004626 | | |
| Wnt14 | AF028702 | | |
| Wnt15 | AF028703 | | |
| Wnt16 | AF169963 | | |
| Receptors | | | |
| FZD1 | NM_003505 | | |
| FZD2 | NM_001466 | tacccagagcggcctatcattttt | 955-1439 |
| | | acgaagccggccaggaggaaggac | |
| FZD3 | NM_017412 | | |
| FZD4 | NM_012193 | | |
| FZD5 | NM_003468 | | |
| FZD6 | NM_003506 | tggcctgaggagcttgaatgtgac | 607-1026 |
| | | atcgcccagcaaaaatccaatgaa | |
| FZD7 | NM_003507 | | |
| FZD8 | AA481448 | | |
| FZD9 | NM_003508 | | |
| FZD10 | NM_007197 | | |
| FRZB | NM_001463 | | |
| Extra- | | | |
| cellular | | | |
| Effectors | | | |
| SFRP1 | NM_003012 | | |
| SFRP2 | AFO 17986 | | |
| SFRP4 | AF026692 | agaggagtggctgcaatgaggtc | 877-1178 |
| | | gcgcccggctgttttctt | |
| SFRP5 | NM_003015 | | |
| SK | AB020315 | | |
| CER1 | NM_005454 | | |
| WIF-1 | NM_007191 | | |
| DVL1 | U46461 | | |
| DVL2 | NM_004422 | | |
| DVL3 | NM_004423 | | |
| Tran- | | | |
| scription | | | |
| Factors | | | |
| Oct4 | Z11899 | taatacgactcactatagggagcag | |
| | | cttgggctcgagaag | |
| | | taatacgactcactatagggccctttg | |
| | | tgttcccaattcc | |
| Brachyury | NM_003181 | | |
| NeuroD1 | NM_002500 | | |
| NeuroD2 | NM_006160 | | |
| NeuroD3 | U63842 | | |
| MyoD | NM_002478 | | |
| MDFL | NM_005586 | | |
| REST | NM_005612 | | |

TABLE 5
Mean Fluorescence Intensity of SSEA-3 (+) and SSEA-3 (−)
(M1 and M2) subpopulations of NTERA2 cells treated with dsRNA,
as described in the legend to FIG. 3

| | Mean Fluorescence Intensity | |
|---|---|---|
| | (Log scale, Arbitary Units) | |
| Treatment | M1 = SSEA3 (+) | M2 = SSEA3 (−) |
| Mock (control) | 319 | 2.0 |
| RNAi (Notch 1 + Notch 2) | 195 | 1.7 |
| RNAi (RBPJk) | 267 | 1.8 |
| RNAi (Oct4) | 181 | 1.6 |
| RNAi control | 354 | 1.7 |

References

Andrews P. W., Goodfellow P. N., Shevinsky L., Bronson D. L. and Knowles B. B. 1982. Cell surface antigens of a clonal human embryonal carcinoma cell line: Morphological and antigenic differentiation in culture. Int. J. Cancer. 29: 523-531.

Andrews P. W., Banting G. S., Damjanov I., Arnaud D. and Avner P. 1984a. Three monoclonal antibodies defining distinct differentiation antigens associated with different high molecular weight polypeptides on the surface of human embryonal carcinoma cells. Hybridoma. 3: 347-361.

Andrews P. W., Damjanov I., Simon D., Banting G., Carlin C., Dracopoli N. C. and Fogh J. 1984b. Pluripotent embryonal carcinoma clones derived from the human teratocarcinoma cell line Tera-2: Differentiation in vivo and in vitro. Lab. Invest. 50: 147-162.

Andrews P. W., Nudelnian E., Hakomori S. -i. and Fenderson B. A. 1990. Different patterns of glycolipid antigens are expressed following differentiation of TERA-2 human embryonal carcinoma cells induced by retinoic acid, hexamethylene bisacetamide (HMBA) or bromodeoxyuridine (BUdR). Differentiation. 43: 131-138.

Fenderson B. A., Andrews P. W., Nudelman E., Clausen H. and Hakomori S.-i. 1987. Glycolipid core structure switching from globo- to lacto- and ganglio-series during retinoic acid-induced differentiation of TERA-2-derived human embryonal carcinoma cells. Dev. Biol. 122: 21-34.

Kannagi, R., Levery, S. B., Ishigami, F., Hakomori, S., Shevinsky, L. H., Knowles, B. B. and Solter, D. (1983) New globoseries glycosphingolipids in human teratocarcinoma reactive with the monoclonal antibody directed to a developmentally regulated antigen, stage-specific embryonic antigen 3. J. Biol. Chem. 258, 8934-8942.

Shevinsky, L. H., Knowles, B. B., Damjanov, I. and Solter, D. (1982) Monoclonal antibody to murine embryos defines a stage-specific embryonic antigen expressed on mouse embryos and human teratocarcinoma cells. Cell 30, 697-705.

Solter, D. and Knowles, B. B. (1978) Monoclonal antibody defining a stage-specific mouse embryonic antigen (SSEA-1). Proc. natl. Acad. Sci. USA 75, 5565-5569.

Recent progress in identifying genes regulating hematopoietic stem cell function and fate Craig T Jordan, Gary Van Zant Current Opinion in Cell Biology 1998, 10:716-720.

Singer M J, Selker E U. Genetic and epigenetic inactivation of repetitive sequences in Neurospora crassa: RIP, DNA methylation, and quelling. Curr Top Microbiol Immunol. 1995;197:165-77.

Matzke M A, Matzke A J. Gene silencing in plants: relevance for genome evolution and the acquisition of genomic methylation patterns. Novartis Found Symp. 1998;214:168-80; discussion 181-6. Review.

Stam M, de Bruin R, van Blokland R, van der Hoom R A, Mol J N, Kooter J M. Distinct features of post-transcriptional gene silencing by antisense transgenes in single copy and inverted T-DNA repeat loci. Plant J. January 2000;21(1):27-42.

Montgomery M K, Xu S, Fire A. RNA as a target of double-stranded RNA-mediated genetic interference in Caenorhabditis elegans. Proc Natl Acad Sci USA. Dec. 22, 1998;95(26):15502-7.

Fire A, Xu S, Montgomery M K, Kostas S A, Driver S E, Mello C C. Potent and specific genetic interference by double-stranded RNA in Caenorhabditis elegans. Nature. Feb. 19, 1998;391(6669):806-11.

Kennerdell J R, Carthew R W. Heritable gene silencing in Drosophila using double-stranded RNA. Nat Biotechnol. August 2000;18(8):896-898.

Shi H, Djikeng A, Mark T, Wirtz E, Tschudi C, Ullu E. Genetic interference in Trypanosoma brucei by heritable and inducible double-stranded RNA. RNA. July 2000;6(7): 1069-76.

Wianny F, Zernicka-Goetz M. Specific interference with gene function by double-stranded RNA in early mouse development. Nat Cell Biol. February 2000;2(2):70-5.

Thomson J A, Itskovitz-Eldor J, Shapiro S S, Waknitz M A, Swiergiel J J, Marshall V S, Jones J M. Embryonic stem cell lines derived from human blastocysts. Science. Nov. 6, 1998 ;282(5391):1145-7.

Thomson J A, Kalishman J, Golos T G, Durning M, Harris C P, Becker R A, Hearn J P. Isolation of a primate embryonic stem cell line. Proc Natl Acad Sci USA. Aug. 15, 1995;92(17):7844-8.

Prober J M, Trainor G L, Dam R J, Hobbs F W, Robertson C W, Zagursky R J, Cocuzza A J, Jensen M A, Baumeister K. A system for rapid DNA sequencing with fluorescent chain-terminating dideoxynucleotides. Science. Oct. 16, 1987;238(4825):336-41.

Feinberg A P, Vogelstein B. A technique for radiolabeling DNA restriction endonuclease fragments to high specific activity. Anal Biochem. Jul. 1, 1983;132(l):6-13.

Mullis K B, Faloona F A. Specific synthesis of DNA in vitro via a polymerase-catalyzed chain reaction. Methods Enzymol. 1987;155:335-50.

Scholer H R, Hatzopoulos A K, Balling R, Suzuki N, Gruss P. A family of octamer-specific proteins present during mouse embryogenesis: evidence for germline-specific expression of an Oct factor. EMBO J. September 1989;8(9):2543-50.

Kraft H J, Mosselman S, Smits H A, Hohenstein P, Piek E, Chen Q, Artzt K, van Zoelen E J. Oct-4 regulates alternative platelet-derived growth factor alpha receptor gene promoter in human embryonal carcinoma cells. J Biol Chem. May 31, 1996;271(22):12873-8.

Reubinoff B E, Pera M F, Fong C Y, Trounson A, Bongso A. Embryonic stem cell lines from human blastocysts: somatic differentiation in vitro. Nat Biotechnol. April 2000;18(4):399-404.

Shamblott M J, Axelman J, Wang S, Bugg E M, Littlefield J W, Donovan P J, Blumenthal P D, Huggins G R, Gearhart J D. Derivation of pluripotent stem cells from cultured human primordial germ cells. Proc Natl Acad Sci USA. Nov. 10, 1998;95(23):13726-31.

Clarke D L, Johansson C B, Wilbertz J, Veress B, Nilsson E, Karlstrom H, Lendahl U, Frisen J. Generalized potential of adult neural stem cells. Science. Jun. 2, 2000;288(5471):1660.

1. An in vitro method to modulate the differentiation state of a pluripotential stem cell selected from the group consisting of: an embryonic stem cell, an embryonic germ cell or a teratocarcinoma stem cell comprising:
(i) contacting said stem cell with at least one inhibitory RNA molecule (RNAi) comprising a sequence of a gene, or the effective part thereof, which mediates at least one step in the differentiation of said cell;
(ii) providing conditions conducive to the growth and differentiation of the cell treated in (i) above; and optionally
(iii) maintaining and/or storing the cell in a differentiated state.

2. A method according to claim 1 wherein said stem cell is an embryonic stem cell.

3. A method according to claim 1 wherein said stem cell is an embryonic germ cell.

4. A method according to claim 1 wherein said stem cell is a teratocarcinoma cell.

5. A method according to any of claims 1-4 wherein said cell surface receptor is selected from the group consisting of: human Notch 1(hNotch 1); hNotch 2; hNotch 3; hNotch 4; TLE-1; TLE-2; TLE-3; TLE-4; TCF7; TCF7L1; TCFFL2; TCF3; TCF19; TCF1; mFringe; lFringe; rFringe; sel 1; Numb; Numblike; LNX; FZD1; FZD2; FZD3; FZD4; FZD5; FZD6; FZD7; FZD8; FZD9; FZD10; and FRZB.

6. A method according to any of claims 1-4 wherein said ligand is selected from the group consisting of: D11-1; D113; D114; Dlk-1; Jagged 1; Jagged 2; Wnt 1; Wnt 2; Wnt 2b; Wnt 3; Wnt 3a; Wnt5a; Wnt6; Wnt7a; Wnt7b; Wnt8a; Wnt8b; Wnt10b; Wnt11; Wnt14; and Wnt15.

7. A method according to any of claims 1-4 wherein said gene is selected from the group consisting of: SFRP1; SFRP2; SFRP4; SFRP5; SK; DKK3; CER1; WIF-1; DVL1; DVL2; DVL3; DVL1L1;mFringe; lFringe; rFringe; sel11; Numb; LNX Oct4;NeuroD1; NeuroD2; NeuroD3; Brachyury; MDFI; CBF-1; and CIR.

8. A method according to any of claims 1-7 wherein said gene comprises at least one of the genes identified by the DNA database accession numbers in Table 4.

9. A method according to claim 8 wherein said gene is selected from the group consisting of: DLK1; Oct 4; hNotch 1; hNotch 2; RBPJk; and CIR.

10. A method according to claim 9 wherein said gene is DLK1.

11. A method according to claim 10 wherein the RNAi molecule is derived from the nucleic acid sequence comprising the sequence presented in FIG. 2a.

12. A method according to claim 9 wherein said gene is Oct 4.

13. A method according to claim 12 wherein the RNAi molecule is derived from the nucleic acid sequence comprising the sequence presented in FIG. 2b.

14. A method according to claim 9 wherein said gene is hNotch 1.

15. A method according to claim 14 wherein said RNAi molecule is derived from the nucleic acid sequence comprising the sequence presented in FIG. 2c.

16. A method according to claim 9 wherein said gene is hNotch 2.

17. A method according to claim 16 wherein said RNAi molecule is derived from the nucleic acid sequence comprising the sequence presented in FIG. 2d.

18. A method according to claim 9 wherein said gene is RBBJk.

19. A method according to claim 18 wehrein said RNAi molecule is derived from the nucleic acid sequence comprising the sequence presented in FIG. 2e.

20. A method according to claim 9 wherein said gene is CIR.

21. A method according to claim 20 wherein said RNAi molecule is derived from the nucleic acid sequence comprising the sequence presented in FIG. 2f.

22. A RNAi molecule characterised in that said molecule comprises the coding sequence of at least one gene which mediates at least one step in stem cell differentiation.

23. A RNAi molecule according to claim 22 wherein said coding sequence is an exon.

24. A RNAi molecule according to claim 22 or 23 wherein said molecule is between 100 bp-1000 bp in length.

25. A RNAi molecule according to claim 24 wherein the length of said molecule is selected from the group consisting of: 100 bp; 200 bp; 300 bp; 400 bp; 500 bp; 600 bp; 700 bp; 800 bp; 900 bp; or 1000 bp.

26. A RNAi molecule according to claim 22 or 23 wherein the length of said molecule is at least 1000 bp.

27. A RNAi molecule according to claim 22 or 23 wherein the length of said molecule is between 15 bp and 25 bp.

28. A RNAi molecule according to claim 27 wherein the length of said molecule is 21 bp.

29. A RNAi molecule according to any of claims 22-28 wherein said molecule comprises a sequence identified by the DNA database accession numbers in Table 4.

30. A RNAi molecule according to claim 29 wherein said RNAi is derived from a gene selected from the group consisting of: DLK1; Oct 4; hNotch 1; hNotch 2; RBPJk; and CIR.

31. A RNAi molecule according to claim 30 wherein said RNAi molecule comprises the nucleic acid sequence selected from the group consisting of the nucleic acid sequences presented in FIGS. 2a-2f.

32. A RNAi molecule according to any of claims 22-31 wherein said molecule comprises modified ribonucleotide bases.

33. Use of an isolated DNA molecule comprising a sequence of a gene which mediates at least one step in stem cell differentiation as represented by the DNA accession numbers identified in Table 4 wherein said DNA molecule is operably linked to at least one further DNA molecule capable of promoting transcription of said DNA linked thereto for the manufacture of a composition for use in mediating in vitro stem cell differentiation.

34. Use according to claim 33 wherein said gene is DLK1.

35. An isolated DNA molecule comprising a sequence of a gene which mediates at least one step in stem cell differentiation as represented by the accession numbers identified in Table 4, excluding NM_{—}003836, wherein said DNA molecule is operably linked to at least one further DNA molecule capable of promoting transcription of said DNA linked thereto.

36. An isolated DNA molecule according to claim 35 wherein said DNA molecule is selected from the group consisting of: Oct 4; hNotch 1; hNotch 2; RBPJk; and CIR.

37. An isolated DNA molecule according to claim 36 wherein said molecule comprises a sequence selected from the group consisting of the sequences as represented in FIGS. 2a-2f.

38. An isolated DNA molecule according to any of claims 35-37 wherein said gene is provided with at least two promoters characterised in that said promoters are oriented such that both DNA strands comprising said DNA molecule are transcribed into RNA.

39. A vector including a DNA molecule according to any of claims 35-38.

40. A method to manufacture RNAi molecules comprising:
(i) providing at least one isolated DNA molecule according to any of claims 35-38 or a vector according to claim 39;
(ii) providing reagents and conditions which allow the synthesis of each RNA strand comprising said RNAi molecule; and
(iii) providing conditions which allow each RNA strand to associate over at least part of their length, or at least that part corresponding to the nucleic acid sequence encoding said stem cell gene which mediates stem cell differentiation.

41. A method according to claim 40 wherein said gene is selected from those genes identified by the DNA database accession numbers in Table 4.

42. A method to promote the differentiation of stem cells comprising administering to an animal an effective amount of RNAi according to any of claims 22-32 sufficient to effect differentiation of a target stem cell.

43. A therapeutic composition comprising at least one RNAi molecule according to any of claims 22-32.

44. The use of at least one RNAi molecule according to any of claims 22-32 for the manufacture of a medicament for use in promoting the differentiation of stem cells to provide differentiated cells/tissues to treat diseases where cell/tissues are destroyed by said disease.

45. The use according to claim 44 wherein said disease is selected from the group consisting of: pernicious anemia; stroke, neurodegenerative diseases such as Parkinson's disease, Alzhiemer's disease; coronary heart disease; cirrhosis; and diabetes.

46. A therapeutic composition according to claim 43 or the use according to claim 44 or 45 which further comprises a diluent, carrier or excipient.

47. A therapeutic cell composition comprising a differentiated cell produced by introduction of a RNAi molecule according to any of claims 22-32.

48. A cell obtainable by the method according to any of claims 1-21.

49. A cell obtainable by the method according to claim 48 wherein said cell is selected from the group consisting of: a nerve cell; a muscle cell; liver cell; a kidney cell; a blood cell (eg erythrocyte, CD4+ cell, CD8+ cell; panceatic β cell; epithelial cell (eg lung, gastric, intestinal).

50. A cell culture obtainable by the method according to any of claims 1-21.

51. An organ comprising at least one cell according to claim 48 or 49.
